(19) United States
(12) Patent Application Publication
(10) Pub. No.: US 2024/0003905 A1
(43) Pub. Date: Jan. 4, 2024

(54) METHODS, SYSTEMS, AND COMPUTER READABLE MEDIA FOR MODULATING TEMPERATURE AND PRODUCING ANALYTE IMAGING DATA

(71) Applicant: ARIZONA BOARD OF REGENTS ON BEHALF OF ARIZONA STATE UNIVERSITY, Scottsdale, AZ (US)

(72) Inventors: Shaopeng WANG, Chandler, AZ (US); Rui WANG, Tempe, AZ (US); Pengfei ZHANG, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/212,460

(22) Filed: Jun. 21, 2023

Related U.S. Application Data
(60) Provisional application No. 63/354,890, filed on Jun. 23, 2022.

(51) Int. Cl. G01N 33/68; G01N 33/543

(52) U.S. Cl. CPC G01N 33/6872; G01N 33/54386; G01N 2333/705

(57) ABSTRACT

Provided herein are methods of modulating temperature in detection fields and producing analyte imaging data. In some embodiments, the methods include introducing an incident light toward a second surface of a substrate to induce a plasmonic wave proximal to a first surface of the substrate such that a temperature in a selected heating space within the detection field is substantially uniformly changed to a selected temperature. Additional methods as well as related systems and computer readable media are also provided.

### CROSS-REFERENCE TO RELATED APPLICATIONS

This application claims the benefit of U.S. Provisional Patent Application Ser. No. 63/354,890 filed Jun. 23, 2022, the disclosure of which is incorporated herein by reference.

### STATEMENT OF GOVERNMENT SUPPORT

This invention was made with government support under R01 GM107165 awarded by the National Institutes of Health. The government has certain rights in the invention.

### BACKGROUND

Temperature is a fundamental environmental parameter that is important in cellular activity regulations. The classical temperature control techniques employ heating sources to conduct the heat transfer, which can be time-consuming and nonuniform over the target surface. In recent decades, plasmon resonances in nanostructures, especially metallic nanoparticles, have been proved to be efficient in regulating localized heat rapidly and feasibly. These nanometer-sized heaters are capable of harvesting light due to the internal decay of hot carriers, facilitating many practical applications, such as photothermal therapy, neuron activation, phase separation, gas sensing, and heterogeneous catalysis. However, localized heating achieved by metallic nanoparticles still suffers from poor space precision of heating due to the random distribution of metallic nanoparticles, resulting in bulk heating on the sample. Moreover, the nanocavities created by the tightly positioned metal nanoparticles may also generate excessive heat, leading to overheating on the target.

Therefore, there is a need for methods, and related aspects, for rapid temperature regulation and uniform temperature distribution over detection fields in various analytical applications.

### SUMMARY

This present disclosure provides rapid temperature modulation processes using plasmonic scattering microscopy. The temperature modulation strategies of the present disclosure have many applications, including activities associated with cellular membrane temperature changes as part of drug screening processes and the like. These and other aspects will be apparent upon a complete review of the present disclosure, including the accompanying figures.

According to various embodiments, a method of modulating temperature in a detection field is presented. The method includes introducing an incident light toward a second surface of a substrate to induce a plasmonic wave at least proximal to a first surface of the substrate such that a temperature in a selected heating space within the detection field is substantially uniformly changed. The first surface of the substrate is coated with a metallic layer. In addition, the selected heating space comprises a Z-dimension that extends above the metallic layer about 110 nm or less, thereby modulating the temperature in the detection field.

According to various embodiments, a system for modulating temperature in a detection field is presented. The system includes a substrate receiving area configured to receive a substrate that comprises first and second surfaces, wherein the second surface is coated with a metallic layer that is configured to create surface plasmon resonance when incident light is introduced toward the second surface at a suitable incident angle via the first surface of the substrate, and wherein the metallic layer comprises at least a first set of analyte binding moieties; a light source configured to introduce an incident light toward the substrate receiving area; a detector configured to collect light scattered by at least one analyte disposed on the metallic layer when the substrate is received in the substrate receiving area and the incident light is introduced from the light source; and a controller that comprises, or is capable of accessing, computer readable media comprising non-transitory computer-executable instructions which, when executed by at least one electronic processor, perform at least: disposing a fluidic sample that comprises the analyte on the second surface of the substrate such that at least a portion of the analyte binds to at least a portion of the first set of analyte binding moieties to produce one or more surface-bound analytes when the substrate is received in the substrate receiving area; introducing the incident light from the light source at the suitable incident angle toward the second surface of the substrate when the substrate is received in the substrate receiving area; introducing the incident light toward the second surface of the substrate such that an area defined by X- and Y-dimensions of a selected heating space within the detection field disposed at least proximal to the second surface of the substrate is within a range of about 1 to about 1000 μm²; adjusting a power density of the incident light such that a temperature in the selected heating space within the detection field is substantially uniformly changed to a selected temperature; and, detecting light scattered by the surface-bound analytes over a duration to produce an analyte imaging data set to thereby at least detect the surface-bound analytes using the detector when the substrate is received in the substrate receiving area. In some embodiments, a fluidic device comprises the substrate. In some embodiments, the fluidic material is substantially free of plasmonic metallic nanoparticles when the fluidic sample that comprises the analyte is disposed on the second surface of the substrate.

According to various embodiments, a computer readable media is presented. The computer readable media comprises non-transitory computer-executable instructions which, when executed by at least one electronic processor, perform at least: disposing a fluidic sample that comprises an analyte on a second surface of a substrate such that at least a portion of the analyte binds to at least a portion of a first set of analyte binding moieties to produce one or more surface-bound analytes when the substrate is received in a substrate receiving area; introducing incident light from a light source at a suitable incident angle toward the second surface of the substrate to create surface plasmon resonance when the substrate is received in the substrate receiving area; introducing the incident light toward the second surface of the substrate such that an area defined by X- and Y-dimensions of a selected heating space within a detection field disposed at least proximal to the second surface of the substrate is within a range of about 1 to about 1000 μm²; adjusting a power density of the incident light such that a temperature in the selected heating space within the detection field is substantially uniformly changed to a selected temperature; and, detecting light scattered by the surface-bound analytes over a duration to produce an analyte imaging data set to thereby at least detect the surface-bound analytes using the detector when the substrate is received in the substrate receiving area.

Various optional features of the above embodiments include the following. A temperature within the detection field that is outside of the selected heating space is substantially unchanged. The method includes flowing a fluidic material over the first surface of the substrate in the selected heating space, which fluidic material is substantially free of plasmonic metallic nanoparticles. The Z-dimension extends above the metallic layer about 100 nm. The selected heating space comprises X- and Y-dimensions and the method comprises introducing the incident light toward the second surface of the substrate such that an area defined by the X- and Y-dimensions of the selected heating space is within a range of about 1 to about 1000 μm². The method includes changing a focus level of the incident light to adjust the area defined by the X- and Y-dimensions of the selected heating space within the range of about 1 to about 1000 μm². The metallic layer comprises gold (Au). The selected heating space comprises at least one analyte and the method comprises detecting light scattered by the analyte to produce an analyte imaging data set. The analyte comprises one or more biomolecules. One or more cells comprise the biomolecules. The biomolecules comprise transient receptor potential vanilloid 1 (TRPV1) ion channels. The analyte comprises one or more fluorescent labels and wherein the method further comprises detecting fluorescent light emitted from the analyte. The method includes adjusting a power density of the incident light such that the temperature in the selected heating space within the detection field is substantially uniformly changed to a selected temperature. The power density of the incident light is no more than about 3 kW/cm². The selected temperature is in a range of about 33° C. to about 80° C. The incident light comprises is 660 nm p-polarized light.

### BRIEF DESCRIPTION OF DRAWINGS

FIGS. 1A and 1B schematically show an exemplary objective-based plasmonic imaging system that can be used to modulate temperature in a detection field according to some aspects disclosed herein. FIG. 1B is a more detailed view of objective-based plasmonic imaging system 100 shown in FIG. 1A.

FIG. 2 is a flow chart that schematically shows exemplary method steps of modulating temperature in a detection field according to some aspects disclosed herein.

FIG. 3 is a schematic diagram of an exemplary system suitable for use with certain aspects disclosed herein.

FIGS. 4A-4D. (A) Schematic overview of the experimental set-up of W-PTM. The thickness of the flow channel is ˜100 μm. The p-polarized 660 nm laser was directed to the Au chip to reach SPR. (B) W-PTM image of roughness scattering from Au chip itself under 1.33 kW/cm². (C) W-PTM images of excitation power-controlled multiple switching process of polymer phase transition. 1 mg/mL HPC (LCST=54° C.) was flowed to the Au chip at a rate of 0.5 mL/mL. Excitation power densities for ‘on’ and ‘off’ states were 2 kW/cm² and 1.33 kW/cm², respectively. Scale bar, 5 μm. (D) Corresponding ensemble W-PTM intensity change in (C). The lower and upper arrows indicate the switch of the laser power density between 2 kW/cm² and 1.33 kW/cm², respectively.

FIGS. 5A-5C. (A, B) Ensemble W-PTM intensity as a function of excitation time of four groups of LCST polymers (33° C. LCST, 45° C. LCST, 62° C. LCST, 72° C. LCST) on three Au chips independently. The excitation power density of (A) and (B) is 1.33 kW/cm² and 3 kW/cm², respectively. (C) Localized equilibrium temperature against power density calibrated by various LCST polymers. The fitted curve shows an exponential relationship between equilibrium temperature and excitation power density (r 2=0.995).

FIGS. 6A-6I. (A) Single-particle analysis processes of W-PTM data. (B, D, F, H) Cumulative particles numbers versus size and time of 33° C. LCST (B), 45° C. LCST (D), 62° C. LCST (F) and 72° C. LCST (H). The excitation power density for (B, D) and (F, H) is 1.33 and 3 kW/cm², respectively. (C, E, G, I) The size histograms of (B, D, F, H) at 100 s, respectively. The solid lines are Gaussian fittings for phase transition generated nanoparticles.

FIGS. 7A-7F. (A) A sketch of imaging set-up for selective TRPV1 activation monitoring. (B) An overlap of the FL image and the W-PTM image, showing the locations of activated cells (lower oval) and un-activated cells (upper oval). (C, E) The FL imaging snapshots (pseudo-color (greyscale)) of cells excited at 0.5 kW/cm² (C) and 1.2 kW/cm² (E). Cells of interest in the W-PTM view and out of W-PTM view were marked with different greyscale squares, respectively. (D, F) the corresponding FL intensity traces of the cells of interest in (C, E), respectively. The arrow marked the time when W-PTM excitation started.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and devices, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Binding: As used herein, the term “binding”, typically refers to a non-covalent association between or among two or more entities.

Detect: As used herein, “detect,” “detecting,” or “detection” refers to an act of determining the existence or presence of one or more analytes in a given sample.

Binding Moiety: As used herein, “binding moiety” refers to a molecule or compound that is capable of binding to an analyte, for example, via a protein or other biomolecule displayed on a surface of a cell.

In some embodiments: As used herein, the term “in some embodiments” refers to embodiments of all aspects of the disclosure, unless the context clearly indicates otherwise.

Moiety: As used herein, “moiety” in the context of chemical compounds or structures refers to one of the portions into which the compound or structure is or can be divided (e.g., a functional group, a substituent group, or the like).

Sample: As used herein, “sample” or “fluidic sample” refers to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide), which is assayed as described herein. A sample may also be any body fluid or excretion (for example, but not limited to, blood, urine, stool, saliva, tears, bile) that contains cells, cell components, or non-cellular fractions.

Subject: As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, terms “individual” or “patient” are used and are intended to be interchangeable with “subject.”

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

System: As used herein, “system” in the context of analytical instrumentation refers a group of objects and/or devices that form a network for performing a desired objective.

### DETAILED DESCRIPTION

Plasmonic absorption of light can create significant local heat and has become a promising tool for rapid temperature regulation. Current plasmonic heating usually relies on specially designed nanomaterials randomly distributed in the space and hardly provides uniform temperature regulation in a wide field. In some embodiments, the technology provided in the present disclosure is a rapid temperature regulation strategy on a plain gold-coated glass slip using plasmonic scattering microscopy, which can be referred to as wide-field plasmonic thermal microscopy (W-PTM). W-PTM has nondestructive local temperature-regulating and concurrent fluorescence imaging capability, and can be a powerful tool to study cellular activities associated with cellular membrane temperature changes.

In some embodiments, W-PTM can provide a temperature regulation range of 33-80° C. at nanometer scale. In some embodiments, W-PTM provides imaging capability, thus allowing statistical analysis of phase-transitioned polymeric nanoparticles, including for drug screening and discovery applications. In some embodiments, W-PTM can be used for noninvasive and local regulation of the transient receptor potential vanilloid 1 (TRPV1) ion channels in the living cells, which can be monitored by simultaneous fluorescence imaging of calcium influx. In some embodiments, W-PTM does not record the propagating plasmonic waves with long decaying length along the surface into the images, thus providing high spatial resolution and Gaussian-distributed point spread function for automatic image processing with, for example, conventional open-source software. In some embodiments, W-PTM enables easy monitoring of thermal dynamics in the time domain by tracking the formation of polymeric aggregation. In some embodiments, W-PTM does not record the strong reflection, thus allowing the incident intensity up to about 3 kW/cm² so that a wide temperature regulation range can be achieved from room temperature to ˜80° C. In some embodiments, W-PTM records the light from the top of the gold surface, making it possible to integrate with fluorescence detection approaches. In some embodiments, the experimental set-up of W-PTM includes a flow channel thickness of about ˜100 μm in which a p-polarized 660 nm laser is directed to a gold coated (Au) chip to reach SPR.

In some embodiments, the systems and methods described herein include implementations of near field optical imaging in which the near field is created by surface plasmon resonance (SPR) or total internal reflection (TIF). Rather than detection of reflected light, however, scattered light from the sample molecules and sensor surface is detected. Light scattered by a molecule in free space scales with the 6th power of the molecular diameter. For this reason, the scattered light intensity diminishes quickly with the molecular size, making it difficult to image single molecules. To overcome this issue, a sensor surface with a selected roughness is used, such that the sensor surface scatters light with a magnitude comparable with that of the scattered light from the target single molecules. There are different ways to define surface roughness, and one of which is given by

$$\sqrt{\frac{1}{n} {\sum }_{i = 1}^{n} {y}_{i}^{2}} \qquad ( 1 )$$

where yᵢ is the height at position i, and n is the number of positions. Using this definition, the surface roughness of a gold surface is −1.5 nm.

In some implementations, a roughness of the sensor surface is in a range of about 1 nm to about 100 nm. The interference of light scattered from the protein and sensor surface produces an image contrast that scales with the 3 rd power of the molecular diameter. This slows down the decay in image contrast with the molecular size, which favors imaging of small objects (e.g., single cells, single protein molecules, etc.).

Rough features and impurities on the sensor surface, and features associated with imperfect optics, all contribute to image contrast, which can mask weak images of single cells or molecules. As described herein, a differential-integral imaging processing algorithm is used to subtract out background features that contribute to image contrast above from each frame of the time sequence images and integrate the differential images to recover the binding and unbinding of single cells on the sensor surface. A drift or motion correction algorithm is introduced to track the drift or motion pattern of one or more features on the sensor surface and correct the drift or motion from each image frame, thereby reducing the impact of drift in position of the sensor surface or the optics or mechanical vibrations of the environment. Binding kinetics are assessed by counting the individual cells on the sensor surface. This digital counting approach allows a precise measurement of binding kinetics. In addition, this approach obviates the need to measure the shift in the surface plasmon resonance angle (determined not only the number of the cells that bind to the sensor surface, but also by the size of the cells) either directly or indirectly.

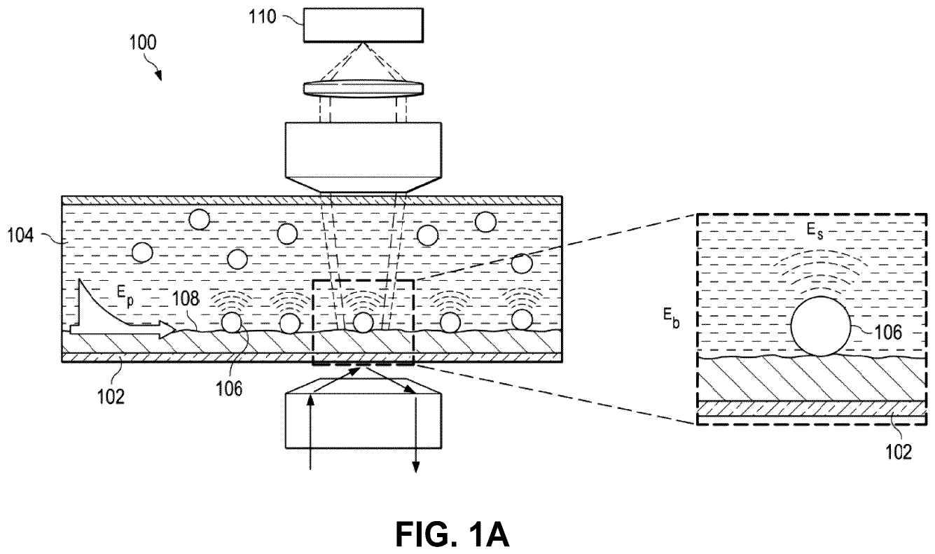

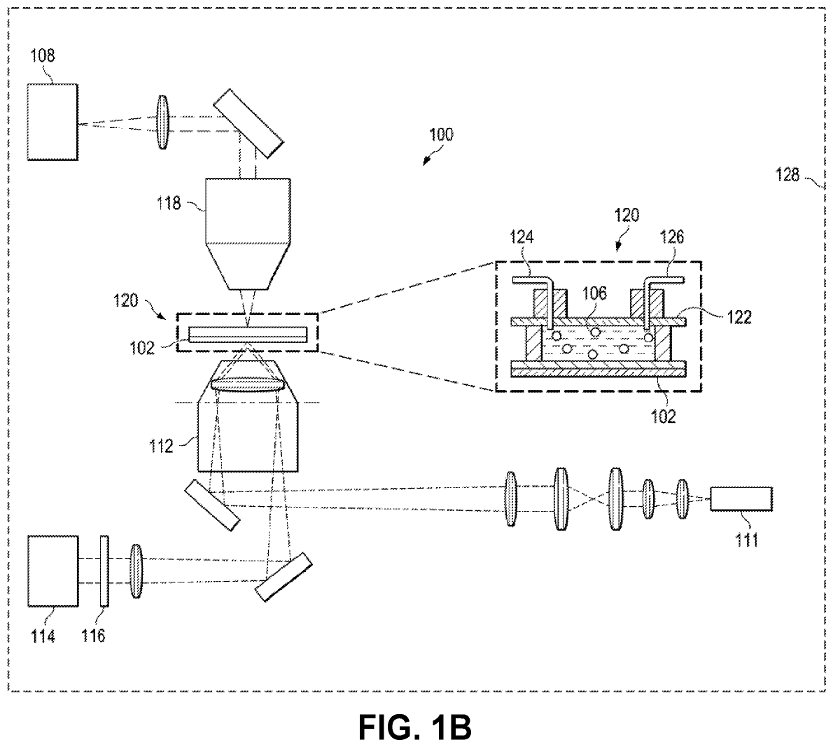

FIG. 1A is a schematic of objective-based plasmonic imaging system 100 that can be used to detect single cell binding to the surface of a sensor. Surface plasmonic waves (Eₚ) are excited by light from the bottom of a gold-coated glass slide and scattering of the plasmonic waves by a particle or exosome (Eₛ) and by the gold surface (E_{b}) is collected from the top to form a plasmonic scattering microscopy (PSM) image. In one example, a sensor includes a metal (e.g., gold) coated glass substrate 102. A solution 104 of the target cell 106 is introduced to the sensing surface (e.g., via a flow cell). The sensor surface can be functionalized with exosome binding moieties 108 for detection of target cells. The light scattered from the cells is collected from the top camera 110. The conventional surface plasmon resonance image can be obtained from a bottom camera simultaneously.

In some implementations, the objective of the system in FIG. 1A is replaced with an optical prism. The prism has a top surface on which the sensor is placed. The prism also has a flat surface for the introduction of incident light and a second flat surface for light reflected from the sensor surface to exit the prism.

FIG. 1B is a more detailed view of objective-based plasmonic imaging system 100. Optical setup for simultaneous PSM and SPR imaging, where light from a super luminescent diode (SLD) 111 is conditioned and directed via a 60× objective (NA=1.49) 112 onto a gold-coated glass slide 102 mounted on the objective via refractive index matching oil. Light reflected from the gold-coated glass slide 102 is detected by camera 114 (Pike F-032B), which is equipped with an optical attenuator 116 (ND30A, Thorlabs, Newton, NJ) to avoid overexposure. The incident light angle is adjusted to surface plasmon resonance, at which the reflected light reaches a minimum. Simultaneously, light scattered from the gold surface is collected by a 50× objective (NA=0.42) 118 and detected by camera 108 (MQ003MG-CM, XIMEA) placed on top of the gold surface. The incident light intensity is 3 kW/cm² or less. Camera 114 measures the traditional SPR and camera 108 records PSM images. Flow cell 120 includes gold-coated glass slide 102, cover glass 122, inlet 124, and outlet 126. In one embodiment, a distance between gold-coated glass slide 102 and cover glass 122 is about 50 microns. However, this distance can be different in other embodiments.

System 100 can include controller 128. Controller 128 can be configured to control one or more components of system 100 (e.g., cameras 108, 114, SLD 111), to control fluid flow to and away from system 100, and to process data or images collected one or more components of system 100 (e.g., cameras 108, 114). In some cases, controller 128 can be used to correct for mechanical drift in system 100.

In one example, gold-coated glass slides were prepared by evaporating 2 nm thick chromium on BK-7 glass slides, followed by 47 nm gold. Before loading into the vacuum chamber for chromium and gold evaporation, the BK-7 glass slides were cleaned by acetone and by deionized water thoroughly. The gold surfaces were examined by Atomic Force Microscopy (AFM), showing islands of variable sizes.

The SPR imaging system has several unique features. First, the evanescent field intensity is localized within −100 nm from the SPR sensor surface (e.g., gold-coated glass slide), making it immune to interference of molecules and impurities in the bulk solution, thus particularly suitable for studying surface binding. Second, there is a large enhancement (20-30 times) in the field near the sensor surface, which is responsible for the high sensitivity of SPR. Finally, the resonance condition of SPR depends on the refractive index near the sensor surface, such that surface charging, small molecules or ions, and biochemical reactions that do not scatter light strongly can also be measured with the same setup from the simultaneously recorded traditional SPR images.

Referring to FIG. 1A, surface plasmonic waves are excited by directing light at an appropriate angle via an oil-immersion objective onto a gold-coated glass slide placed on the objective. In traditional SPR, light reflected from the gold surface is collected to form an SPR image, which is described by

I˜|Eᵥ+Eₑ+E^{r}|², (2)

where Eₚ is the plasmonic wave excited by the incident light, Eₛ describes the scattering of the plasmonic wave by an exosome on the sensor surface, and Eᵣ is the reflection of the incident wave from the backside of the gold surface. The SPR image contrast is determined by the interference between the planar plasmonic wave and the spherical scattered plasmonic wave, given by 2|Eₚ∥Eₛ|cos(θ), where Q is the phase difference between the two waves, which produces a spot at the location of the exosome with a parabolic tail. Eₛ is proportional to the optical polarizability of the exosome, which scales with the mass of the exosome or d³, where d is the diameter.

Eᵣ in Eq. 2 produces a large background in the SPR image, which masks the weak scattered wave (Eₛ) from a single exosome. To overcome this difficulty, plasmonic waves scattered by the exosome are imaged with a second objective placed on top of the sample, in addition to recording the traditional SPR images from the bottom. This avoids the collection of the strong reflection and also eliminates the parabolic tail, providing a high contrast image of the exosome. At first glance, the image contrast should scale according to |Eₛ|²˜d⁶. This would lead to a rapid drop in the image contrast with decreasing d, making it challenging to detect small exosomes. However, the gold surface is not atomically flat. Atomic Force Microscopy (AFM) has revealed nm-scaled gold islands, which scatter the surface plasmonic waves and produce a background (E_{b}) also collected by the top objective. Consequently, the plasmonic image is given by

I˜|E_{b}+E_{z}|²=|E_{b}|²+2|E_{b}∥Eₛ|cos(β)+|Eₛ|², (3)

where β is the phase difference between light scattered by the exosome and by the gold surface. The interference term, 2|E_{b}∥Eₛ|cos(β), in Eq.3 produces image contrast that scales with d 3, or the mass of the exosome. To differentiate this plasmonic imaging method from the traditional SPR imaging, it is referred to as PSM.

To obtain a high contrast PSM image, |E_{b}|² is removed from Eq. 3, which is achieved with the following imaging processing flow. Starting from the raw images captured with a high frame rate, the image frames are averaged (e.g., over 50 ms) to remove pixel and other random noise in the images. Differential images are then obtained by subtracting a previous frame from each frame, or I(N)−I(N−1), where I(N) and I(N−1) are the N^{th} and (N−1)^{th} image frames. The subtraction removes background features and captures the binding of an exosome to the surface on N^{th} image frame. To view all the exosomes on the surface on N^{th} frame, the differential images are integrated from 1 to N. Due to thermal and mechanical drift of the optical system, a drift correction mechanism is introduced to ensure effective removal of the background.

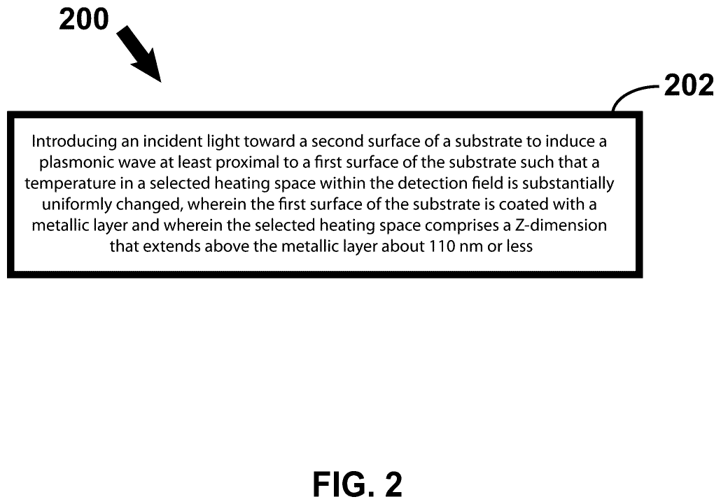

To illustrate, FIG. 2 is a flow chart that schematically shows exemplary method steps of modulating temperature in a detection field. As shown, method 200 includes introducing an incident light toward a second surface of a substrate to induce a plasmonic wave at least proximal to a first surface of the substrate such that a temperature in a selected heating space within the detection field is substantially uniformly changed (step 202). The first surface of the substrate is coated with a metallic layer. The selected heating space comprises a Z-dimension that extends above the metallic layer about 110 nm or less.

In some embodiments, a temperature within the detection field that is outside of the selected heating space is substantially unchanged. In some embodiments, method 200 includes flowing a fluidic material over the first surface of the substrate in the selected heating space, which fluidic material is substantially free of plasmonic metallic nanoparticles. In some embodiments, the Z-dimension extends above the metallic layer about 100 nm. In some embodiments, the selected heating space comprises X- and Y-dimensions and method 200 comprises introducing the incident light toward the second surface of the substrate such that an area defined by the X- and Y-dimensions of the selected heating space is within a range of about 1 to about 1000 μm². In some embodiments, method 200 includes changing a focus level of the incident light to adjust the area defined by the X- and Y-dimensions of the selected heating space within the range of about 1 to about 1000 μm². In some embodiments, the metallic layer comprises gold (Au). In some embodiments, the selected heating space comprises at least one analyte and the method comprises detecting light scattered by the analyte to produce an analyte imaging data set. In some embodiments, the analyte comprises one or more biomolecules. In some embodiments, one or more cells comprise the biomolecules. In some embodiments, the biomolecules comprise transient receptor potential vanilloid 1 (TRPV1) ion channels. In some embodiments, the analyte comprises one or more fluorescent labels and wherein the method further comprises detecting fluorescent light emitted from the analyte. In some embodiments, method 200 includes adjusting a power density of the incident light such that the temperature in the selected heating space within the detection field is substantially uniformly changed to a selected temperature. In some embodiments, the power density of the incident light is no more than about 3 kW/cm². In some embodiments, the selected temperature is in a range of about 33° C. to about 80° C. In some embodiments, the incident light comprises is 660 nm p-polarized light.

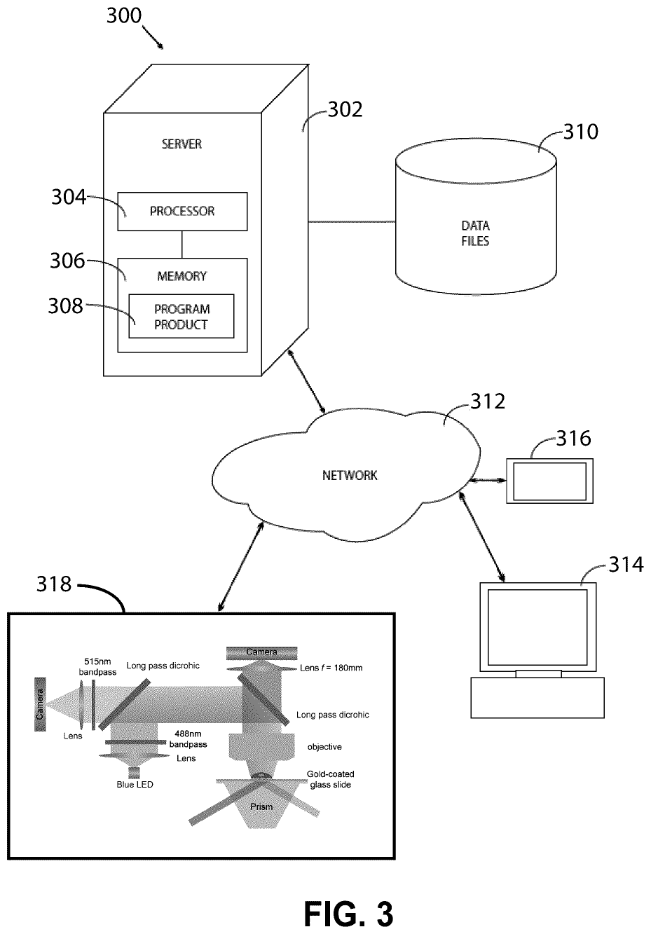

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 3 provides a schematic diagram of an exemplary system suitable for use with implementing at least aspects of the methods disclosed in this application. As shown, system 300 includes at least one controller or computer, e.g., server 302 (e.g., a search engine server), which includes processor 304 and memory, storage device, or memory component 306, and one or more other communication devices 314, 316, (e.g., client-side computer terminals, telephones, tablets, laptops, other mobile devices, etc. (e.g., for receiving imaging data sets or results, etc.) in communication with the remote server 302, through electronic communication network 312, such as the Internet or other internetwork. Communication devices 314, 316 typically include an electronic display (e.g., an internet enabled computer or the like) in communication with, e.g., server 302 computer over network 312 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 300 also includes program product 308 (e.g., for modulating temperature in a detection field as described herein) stored on a computer or machine readable medium, such as, for example, one or more of various types of memory, such as memory 306 of server 302, that is readable by the server 302, to facilitate, for example, a guided search application or other executable by one or more other communication devices, such as 314 (schematically shown as a desktop or personal computer). In some aspects, system 300 optionally also includes at least one database server, such as, for example, server 310 associated with an online website having data stored thereon searchable either directly or through search engine server 302. System 300 optionally also includes one or more other servers positioned remotely from server 302, each of which are optionally associated with one or more database servers 310 located remotely or located local to each of the other servers. The other servers can beneficially provide service to geographically remote users and enhance geographically distributed operations.

As understood by those of ordinary skill in the art, memory 306 of the server 302 optionally includes volatile and/or nonvolatile memory including, for example, RAM, ROM, and magnetic or optical disks, among others. It is also understood by those of ordinary skill in the art that although illustrated as a single server, the illustrated configuration of server 302 is given only by way of example and that other types of servers or computers configured according to various other methodologies or architectures can also be used. Server 302 shown schematically in FIG. 3, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 300. As also understood by those of ordinary skill in the art, other user communication devices 314, 316 in these aspects, for example, can be a laptop, desktop, tablet, personal digital assistant (PDA), cell phone, server, or other types of computers. As known and understood by those of ordinary skill in the art, network 312 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 308 is optionally in the form of microcode, programs, cloud computing format, routines, and/or symbolic languages that provide one or more sets of ordered operations that control the functioning of the hardware and direct its operation. Program product 308, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 308 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 308 is optionally copied from the computer-readable medium to a hard disk or a similar intermediate storage medium. When program product 308, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 308 includes non-transitory computer-executable instructions which, when executed by electronic processor 304, perform at least: disposing a fluidic sample that comprises an analyte on a second surface of a substrate such that at least a portion of the analyte binds to at least a portion of a first set of analyte binding moieties to produce one or more surface-bound analytes when the substrate is received in a substrate receiving area; introducing incident light from a light source at a suitable incident angle toward the second surface of the substrate to create surface plasmon resonance when the substrate is received in the substrate receiving area; introducing the incident light toward the second surface of the substrate such that an area defined by X- and Y-dimensions of a selected heating space within a detection field disposed at least proximal to the second surface of the substrate is within a range of about 1 to about 1000 μm²; adjusting a power density of the incident light such that a temperature in the selected heating space within the detection field is substantially uniformly changed to a selected temperature; and, detecting light scattered by the surface-bound analytes over a duration to produce an analyte imaging data set to thereby at least detect the surface-bound analytes using the detector when the substrate is received in the substrate receiving area.

Typically, imaging is obtained using device or subassembly 318. As shown, device or subassembly 318 includes a gold coated glass slide. Incident light is introduced via a second surface of the slide and light scattered from analyte molecules disposed on the first surface is detected using the cameras.

### Example: Rapid Regulation of Local Temperature and TRPV1 Ion Channels with Wide-Field Plasmonic Thermal Microscopy

Introduction

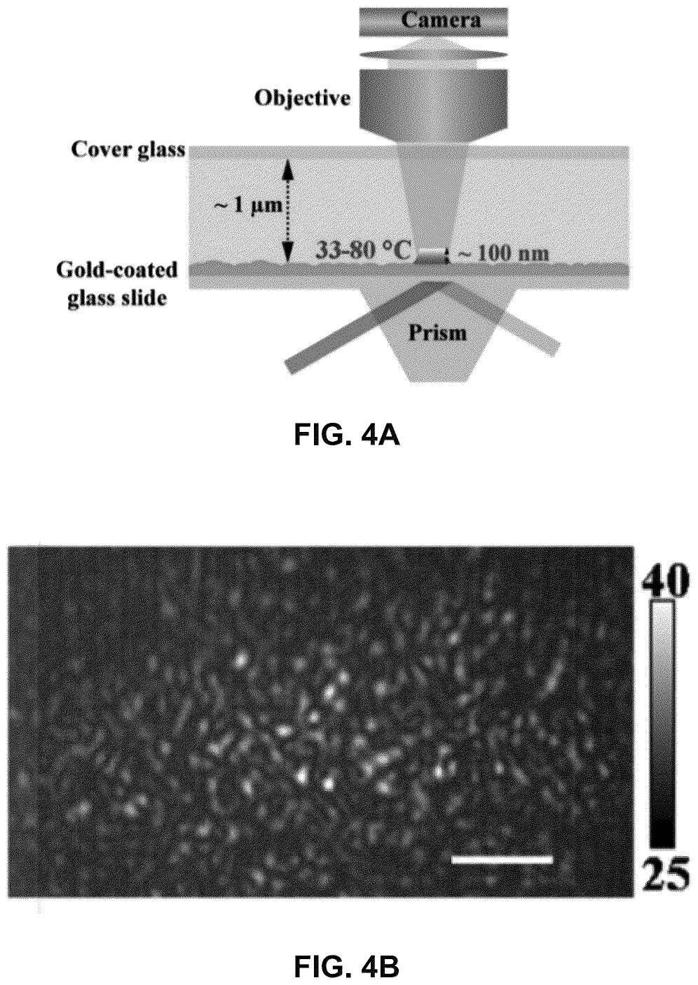

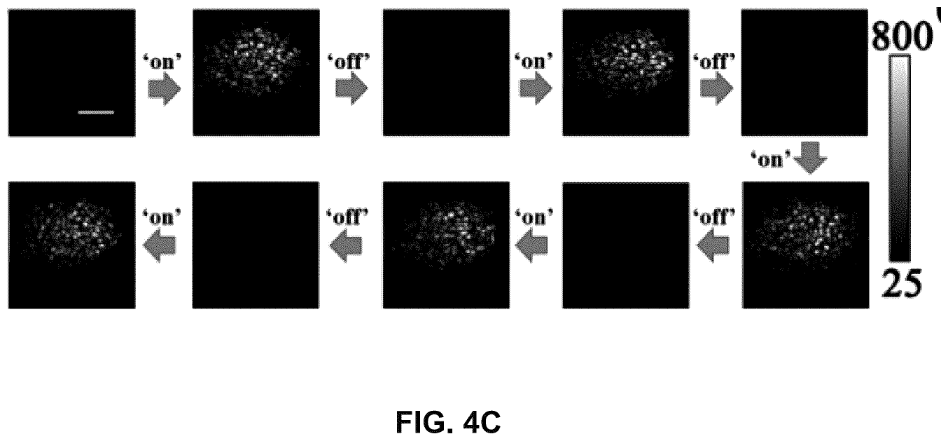

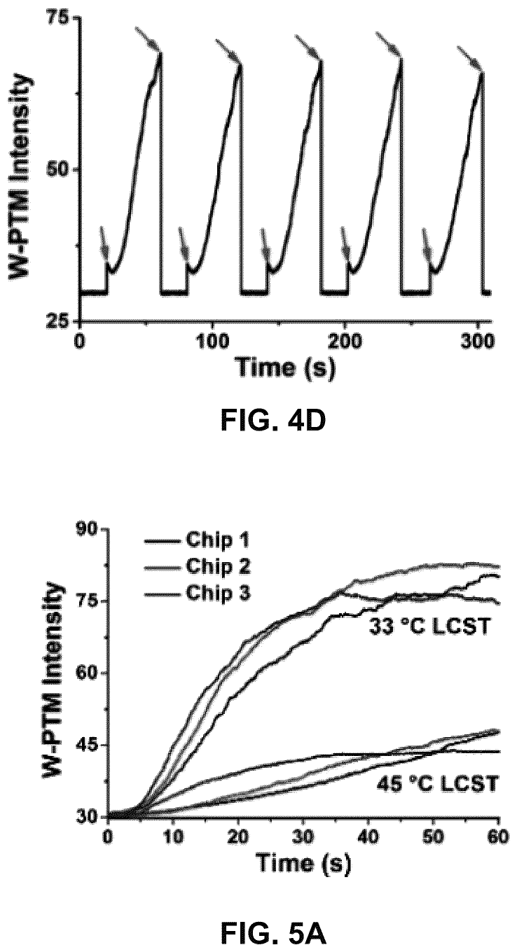

This example shows wide-field plasmonic thermal microscopy (W-PTM), which provides rapid temperature regulation and uniform temperature distribution over its detection field (FIG. 4A). Specifically, W-PTM can utilize the evanescent properties of surface plasmonic waves to limit the heating space within ˜100 nm nearby the gold surface, providing a feasible way to selectively heat the temperature-sensitive membrane proteins, such as the transient receptor potential vanilloid 1 (TRPV1) ion channels, for regulating the cell activities. First, we calibrated the temperature regulation range and temporal dynamics of W-PTM by monitoring the phase transitions of temperature-responsive polymers in aqueous solutions. Then, we employed W-PTM to selectively activate ion channels in TRPV1 transfected HEK-293T cells, accompanied by fluorescence imaging to monitor the intercellular calcium ions influx processes, demonstrating the feasibility of using W-PTM to study the temperature responsiveness of living cells.

The W-PTM was developed based on the plasmonic scattering microscopy (PSM), a novel wide-field surface plasmon resonance (SPR) imaging approach. In addition to the wide-field imaging ability, which is beyond the capabilities of localized SPR detection with metallic nanoparticles, the W-PTM also shares the advantages of PSM over the traditional SPR systems: 1) the W-PTM does not record the propagating plasmonic waves with long decaying length along the surface into the images, thus providing high spatial resolution and Gaussian-distributed point spread function for automatic image processing with conventional open-source software such as ImageJ. As a result, W-PTM enables easy monitoring of thermal dynamics in the time domain by tracking the formation of polymeric aggregation (i.e., phase transition) particles. 2) W-PTM does not record the strong reflection, thus allowing the incident intensity up to 3 kW/cm² so that a wide temperature regulation range can be achieved from room temperature to ˜80° C. 3) W-PTM records the light from the top of the gold surface, making it possible to integrate with fluorescence detection approaches, whose signals are massively dissipated through the gold surface in traditional SPR systems.

To investigate the local heating behavior on W-PTM, polymers with low critical solution temperature (LCST) were chosen due to their phase transition and non-photobleaching features. Moreover, a wide range of responsive temperatures (i.e., LCST) can be easily achieved by applying the salt effect to a specific polymer. Temperature-dependent dynamic light scattering (DLS) results showed that 8 groups of LCST polymer precursor with LCST ranging from 33-80° C. could be achieved by dissolving hydroxypropyl cellulose (HPC) or poly(ethylene oxide)(PEO, Mw=100,000) in Na₂HPO₄ or NaCl aqueous solution with various salt concentrations. As shown in FIG. 4, after applying a specific LCST polymer precursor, such as 1 mg/mL HPC (LCST=54° C.), to the Au chip surface, dynamic formation of bright scattering spots can be observed in the W-PTM image when incident light power above a threshold level, in contrast with weak roughness scattering from Au chip itself (FIG. 4B). We also confirmed that the appearance and disappearance of these bright spots can be switched ‘on’ and ‘off’ reproducibly for 5 times by tuning the excitation power of the W-PTM (FIGS. 4C, 4D). These phenomena suggest that these bright spots shown in the scattering images are from dynamic formation of polymer nanoparticles associated with local temperature change.

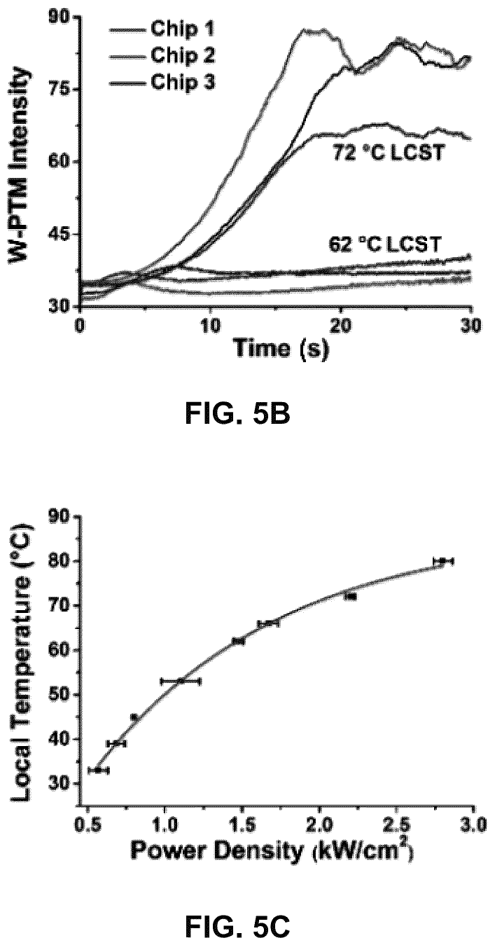

In order to further confirm the feasibility of LCST polymer in sensing the local temperature of W-PTM, we tested the phase transition processes of four LCST polymer precursors (LCST=33, 45, 62, 72° C., referred to as 33° C. LCST, 45° C. LCST, 62° C. LCST, 72° C. LCST, respectively) on three independent Au chips, respectively. By measuring ensemble image intensity as a function of excitation power density, we found that the phase transitions of these LCST polymers are consistent among these chips. Interestingly, HPC-based (LCST=33, 45° C., FIG. 5A) and PEO-based (LCST=62, 72° C., FIG. 5B) LCST polymers show inverse correlation between W-PTM intensity and LCST temperature. Specifically, W-PTM intensity produced by 33° C. LCST is higher than that produced by 45° C. LCST under the same power excitation, while the opposite trend is found on 62° C. LCST and 72° C. LCST. We attribute the former to a higher phase transition threshold energy required for 45° C. LCST than 33° C. LCST and thus resulting in a slower and smaller nanoparticle formation at the same power, which can also be reflected by the slower kinetics of the W-PTM intensity change of 45° C. LCST. However, the ensemble signal analysis cannot explain the difference between 62° C. LCST and 72° C. LCST, for which single-particle analysis is required.

Next, we calibrated the surface temperature of the gold chip using these LCST polymers. We gradually increased the W-PTM excitation power density in a step-by-step manner while monitoring the phase transition of a specific LCST polymer. The W-PTM intensity becomes very sensitive to the excitation power when the local temperature reaches the phase transition temperature, defined by the W-PTM intensity change exceeding five times the fluctuation of the background signal. And its equilibrium temperature was defined as the phase transition temperature of the specific LCST polymer. FIG. 5C shows the local equilibrium temperature under various power densities by measuring these LCST polymers. Typically, local heat would reach equilibrium within 10 s of excitation, reflected by the steady increase of the W-PTM intensity. We found that the local equilibrium temperature is exponentially related to the power density. Our results clearly demonstrate that a local temperature of ˜62° C. can be reached at 1.5 kW/cm², a power density often used in the single-molecule analysis. The localized heat of W-PTM may cause the inactivation of thermally unstable samples, such as proteins, etc. Therefore, our temperature calibration curve helps to find out the up-limit of the power density that can be used to detect heat-sensitive samples.

To validate the above findings, low-density lipoprotein (LDL), a natural heat-sensitive protein with a transition temperature at ˜78° C., was used as a reference. We determined the phase transition power density and the corresponding phase transition time of LDL to be 2.1 kW/cm² and 16 s, respectively, which is comparable to that of 80° C. LCST (2.5 kW/cm² and 19 s). Both LDL and 80° C. LCST showed phase transition behavior when excited at the same power density (3 kW/cm²), but the ensemble W-PTM intensity change of LDL is close to an exponential increase, while that of 80° C. LCST is closer to linear increase. These dynamics can be explained by the slight lower phase transition temperature of LDL making burst nucleation of LDL at this power density, while the 80° C. LCST is just reaching the phase transition temperature and polymerizing at a slower rate. Nevertheless, this result confirms that local temperature calibration using LCST polymer is reliable and accurate.

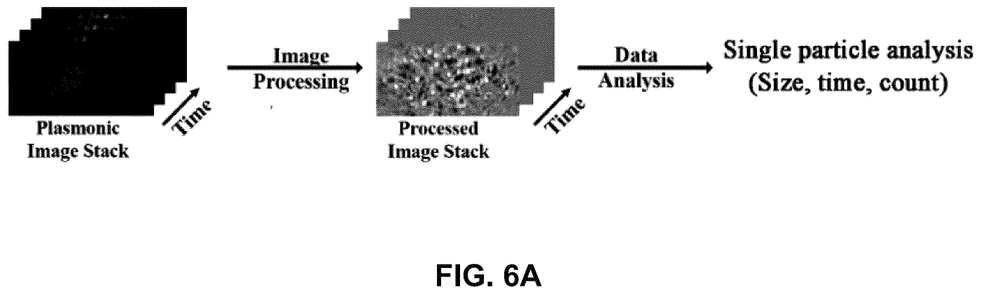

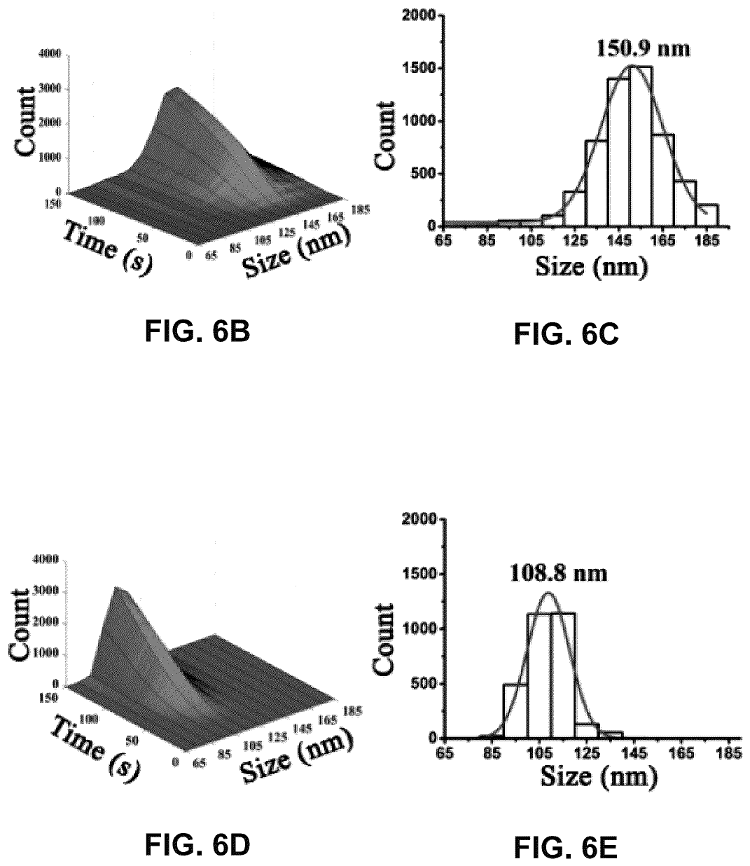

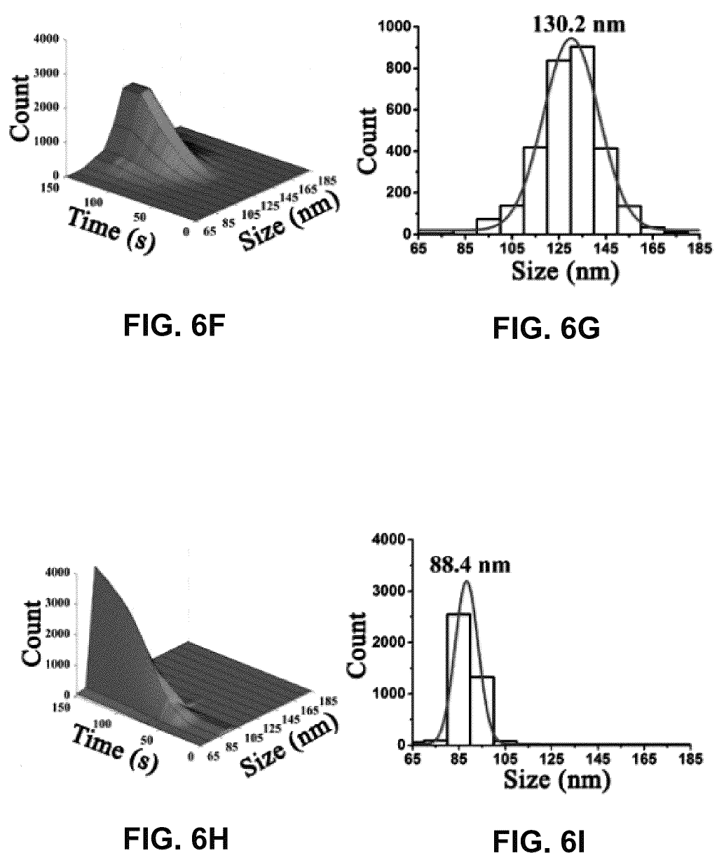

Taking advantage of the single-particle analysis capability of W-PTM, an image processing algorithm was employed to analyze the size change of the generated particles during the phase transition. We first subtract a previous frame from each frame to remove background features, resulting in the differential images (FIG. 6A). Then these differential images were analyzed by Trackmate, a plugin software affiliated with ImageJ, to obtain the showing time, location and W-PTM intensity of each particle. Meanwhile, according to the intensity-size equation we built previously, the W-PTM intensities are correlated with their actual size. By plotting cumulative particle numbers against size and time, we can obtain the phase transition dynamics of LCST polymers at the single-particle level. For example, nanoparticles with a size centered at ˜150.9 nm will appear for 33° C. LCST polymer after excitation at 1.33 kW/cm² for about 20 s, and while the number of particles increases, the particle size remains unchanged during the entire phase transition process (FIGS. 6B, 6C). In addition, we also found that 45° C. LCST (FIGS. 6D, 6E) yielded smaller particle sizes (˜108.8 nm) than 33° C. LCST under the same conditions with similar counts, which is consistent with ensemble measurements on 33° C. LCST and 45° C. LCST (FIG. 4D). In the case of 62° C. LCST and 72° C. LCST, single particle analysis indicated that the former has larger particle size (130.2 nm vs. 88.4 nm) (FIGS. 6F-6I), but 72° C. LCST showed faster particle generation kinetics, which resulted in twice as many particles as 62° C. LCST. This phenomenon can explain why 72° C. LCST has a stronger ensemble W-PTM intensity than 62° C. LCST (FIG. 5B). Overall, single particle analysis can provide more information on the phase transition process than ensemble signal analysis and provide phase transition details at the nanometer scale.

We expect W-PTM to be applied to biological micro-heaters, owing to the advantages of non-invasiveness, label-free feature, accurate temperature control, and light-triggered fast switching feature. As a proof of concept, we attempted to exploit the local thermal effect of W-PTM to selectively activate transient receptor potential vanilloid 1 (TRPV1) ion channels. The TRPV1 ion channel is a calcium-permeable non-selective cation channel that can be activated by capsaicin, heat (>42° C.), pH (<5.9), voltage, and other stimuli. After activated, the action potential can be triggered to promote downstream intracellular signal transduction processes. Thus, elucidating how TRPV1 responds to heat or drugs is vital to understanding diseases that affect every major organ system of the body. However, the activation of TRPV1 is often limited to a few ensemble triggering methods, such as halogen lamps, focused lasers, and drug flows, making it difficult to achieve selective activation.

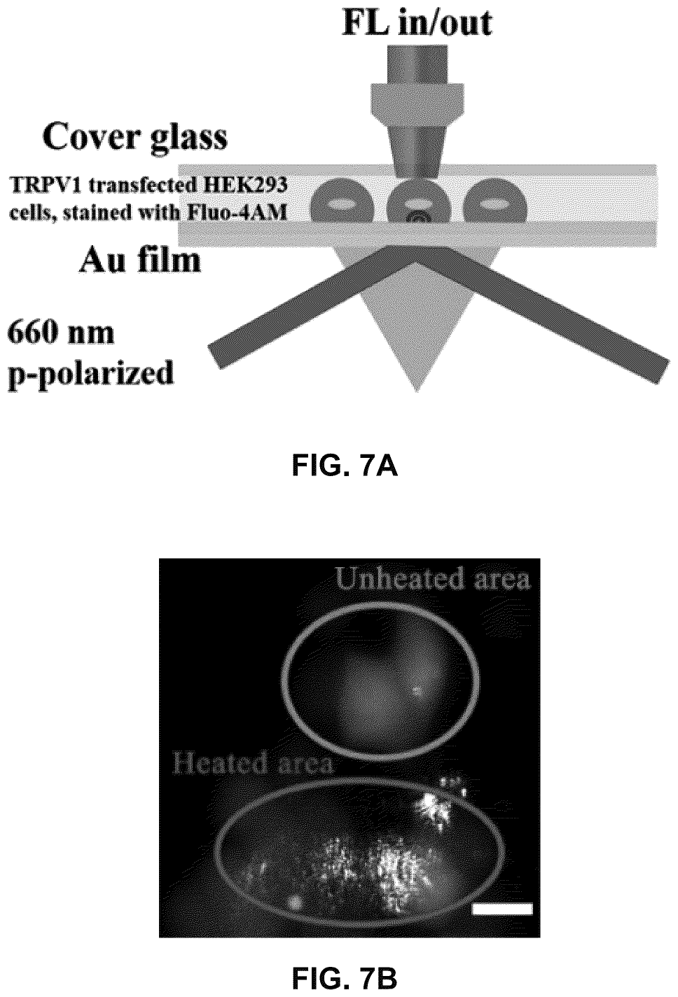

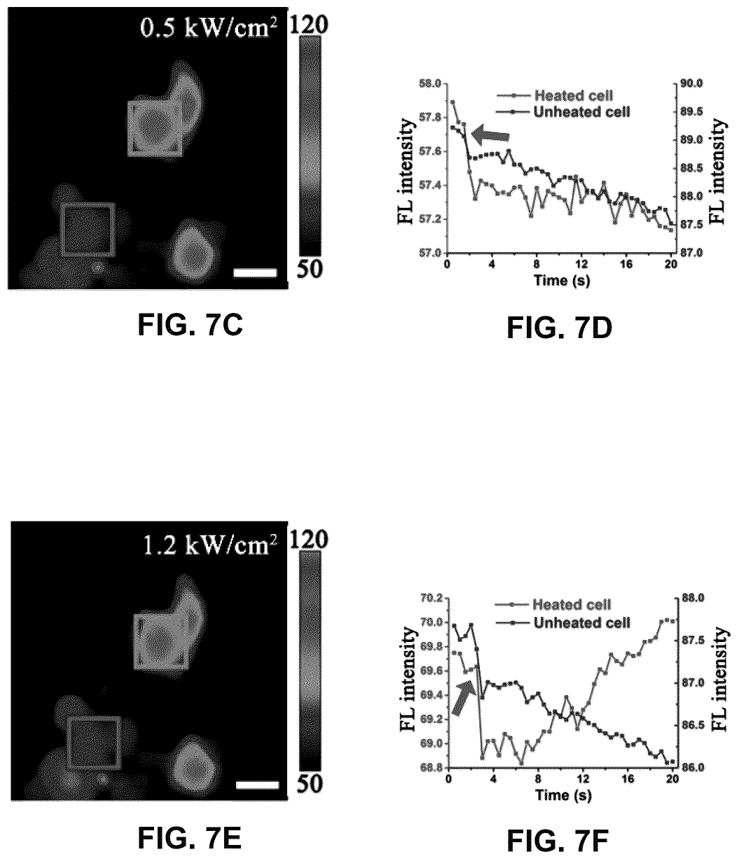

To investigate the response of TRPV1 ion channels to the localized heat of the W-PTM, we employed the calcium imaging by integrating a set of fluorescence (FL) imaging pathways along with W-PTM (FIG. 4a), and the TRPV1-transfected cells were also stained by Fluo-4AM, a calcium indicator dye that responds to TRPV1 activation by changes in FL intensity. From the superposition of the FL image and the W-PTM image (FIG. 7B), we can see that only a part of the TRPV1-transfected cells can be thermally stimulated. Next, we monitored the FL changes of these cells responding to W-PTM excitations. As can be seen in FIG. 7C, we found that when the equilibrium temperature (33° C.) provided by the W-PTM (power density=0.5 kW/cm²) was lower than the activation threshold temperature of TRPV1, both heated and non-heated cells remained un-activated, characterized by a continuous decrease in fluorescence (FIG. 7D), corresponding to the photobleaching of the Fluo-4AM. Then, we further increased the power density of W-PTM to 1.2 kW/cm², with an equilibrium temperature −12° C. higher than the activation threshold of TRPV1. We found that the FL intensity of heated cells showed a sudden drop but increased gradually. We suspect that the sudden drop in FL in heated cells is related to quick photobleaching of Fluo-4AM due to the onset of W-PTM excitation, while the subsequent FL increase can be explained by the large influx of calcium ions accompanying the opening of the TRPV1 ion channel. Meanwhile, the unheated cells remain un-activated, characterized by consistent photobleaching (FIGS. 7E, 7F). These results show that the localized thermal effect of W-PTM can achieve precise and controllable activation of TRPV1 ion channels, which could be used for functional study of TRPV1 and accelerate related drug discovery process.

Compared with the traditional micro-heating methods, such as plasmonic metallic nanoparticles heating, W-PTM provides a more precise and controllable micro-region heating. Owing to the evanescent properties of surface plasmonic waves on W-PTM, the Z-dimension of heating space is limited to ˜100 nm nearby the gold surface, while the XY-dimension can be controlled by the laser focus ranging from ˜1 to hundreds pmt. Moreover, the local temperature on the W-PTM is controllable within 33-80° C. with no overheating effects. Meanwhile, W-PTM can also analyze thermal transition kinetic processes at the single-molecule or single-particle scale, such as the phase transition process of LCST polymers. In addition to quantify the particle size of these generated nanoparticles, W-PTM can also provide 1 ms temporal resolution for rapid real time counting of the particles, better than ensemble measuring methods like DLS.

Until now, the heat activation of TRPV1 is often limited to ensemble triggering methods that lack of selectivity at cell level. In this example, we applied W-PTM in selective heating of the TRPV1 transfected cells, while calcium fluorescence imaging was used as a reference. We demonstrated that W-PTM can selectively activate the cells in the field of view (as few as several cells) in an excitation power-dependent manner without affecting surrounding cells. Our previous study demonstrated that PSM, the parent technique of W-PTM, can image cell deformation at the single focal adhesion level. Therefore, in future studies we could monitor the dynamic cell deformations to characterize the thermally controlled ion channel states. We anticipate that W-PTM will become a powerful tool for studying TRPV1 and other thermal responsive cellular activities, and accelerating drug screening throughput in the future.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, and/or computer readable media or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A method of modulating temperature in a detection field, the method comprising introducing an incident light toward a second surface of a substrate to induce a plasmonic wave at least proximal to a first surface of the substrate such that a temperature in a selected heating space within the detection field is substantially uniformly changed, wherein the first surface of the substrate is coated with a metallic layer and wherein the selected heating space comprises a Z-dimension that extends above the metallic layer about 110 nm or less, thereby modulating the temperature in the detection field.

2. The method of claim 1, wherein a temperature within the detection field that is outside of the selected heating space is substantially unchanged.

3. The method of claim 1, comprising flowing a fluidic material over the first surface of the substrate in the selected heating space, which fluidic material is substantially free of plasmonic metallic nanoparticles.

4. The method of claim 1, wherein the Z-dimension extends above the metallic layer about 100 nm.

5. The method of claim 1, wherein the selected heating space comprises X- and Y-dimensions and wherein the method comprises introducing the incident light toward the second surface of the substrate such that an area defined by the X- and Y-dimensions of the selected heating space is within a range of about 1 to about 1000 μm².

6. The method of claim 1, comprising changing a focus level of the incident light to adjust the area defined by the X- and Y-dimensions of the selected heating space within the range of about 1 to about 1000 μm².

7. The method of claim 1, wherein the metallic layer comprises gold (Au).

8. The method of claim 1, wherein the selected heating space comprises at least one analyte and wherein the method comprises detecting light scattered by the analyte to produce an analyte imaging data set.

9. The method of claim 8, wherein the analyte comprises one or more biomolecules.

10. The method of claim 9, wherein one or more cells comprise the biomolecules.

11. The method of claim 9, wherein the biomolecules comprise transient receptor potential vanilloid 1 (TRPV1) ion channels.

12. The method of claim 8, wherein the analyte comprises one or more fluorescent labels and wherein the method further comprises detecting fluorescent light emitted from the analyte.

13. The method of claim 1, comprising adjusting a power density of the incident light such that the temperature in the selected heating space within the detection field is substantially uniformly changed to a selected temperature.

14. The method of claim 13, wherein the power density of the incident light is no more than about 3 kW/cm².

15. The method of claim 13, wherein the selected temperature is in a range of about 33° C. to about 80° C.

16. The method of claim 1, wherein the incident light comprises is 660 nm p-polarized light.

17. A system for modulating temperature in a detection field, comprising:
a substrate receiving area configured to receive a substrate that comprises first and second surfaces, wherein the second surface is coated with a metallic layer that is configured to create surface plasmon resonance when incident light is introduced toward the second surface at a suitable incident angle via the first surface of the substrate, and wherein the metallic layer comprises at least a first set of analyte binding moieties;
a light source configured to introduce an incident light toward the substrate receiving area;
a detector configured to collect light scattered by at least one analyte disposed on the metallic layer when the substrate is received in the substrate receiving area and the incident light is introduced from the light source; and
a controller that comprises, or is capable of accessing, computer readable media comprising non-transitory computer-executable instructions which, when executed by at least one electronic processor, perform at least:
disposing a fluidic sample that comprises the analyte on the second surface of the substrate such that at least a portion of the analyte binds to at least a portion of the first set of analyte binding moieties to produce one or more surface-bound analytes when the substrate is received in the substrate receiving area;
introducing the incident light from the light source at the suitable incident angle toward the second surface of the substrate when the substrate is received in the substrate receiving area;
introducing the incident light toward the second surface of the substrate such that an area defined by X- and Y-dimensions of a selected heating space within the detection field disposed at least proximal to the second surface of the substrate is within a range of about 1 to about 1000 μm²;
adjusting a power density of the incident light such that a temperature in the selected heating space within the detection field is substantially uniformly changed to a selected temperature; and,
detecting light scattered by the surface-bound analytes over a duration to produce an analyte imaging data set to thereby at least detect the surface-bound analytes using the detector when the substrate is received in the substrate receiving area.

18. The system of claim 17, wherein a fluidic device comprises the substrate.

19. The system of claim 17, wherein the fluidic material is substantially free of plasmonic metallic nanoparticles when the fluidic sample that comprises the analyte is disposed on the second surface of the substrate.

20. A computer readable media comprising non-transitory computer-executable instructions which, when executed by at least one electronic processor, perform at least:
disposing a fluidic sample that comprises an analyte on a second surface of a substrate such that at least a portion of the analyte binds to at least a portion of a first set of analyte binding moieties to produce one or more surface-bound analytes when the substrate is received in a substrate receiving area;
introducing incident light from a light source at a suitable incident angle toward the second surface of the substrate to create surface plasmon resonance when the substrate is received in the substrate receiving area;
introducing the incident light toward the second surface of the substrate such that an area defined by X- and Y-dimensions of a selected heating space within a detection field disposed at least proximal to the second surface of the substrate is within a range of about 1 to about 1000 μm²;
adjusting a power density of the incident light such that a temperature in the selected heating space within the detection field is substantially uniformly changed to a selected temperature; and,
detecting light scattered by the surface-bound analytes over a duration to produce an analyte imaging data set to thereby at least detect the surface-bound analytes using the detector when the substrate is received in the substrate receiving area.
